(19) United States
(12) Patent Application Publication
(10) Pub. No.: US 2025/0041265 A1
(43) Pub. Date: Feb. 6, 2025

(54) NEW DELIVERY SYSTEM FOR VITAMIN C

(71) Applicant: DSM IP Assets B.V., Heerlen (NL)

(72) Inventors: Elger FUNDA, Kaiseraugst (CH); Odile KRAINZ, Kaiseraugst (CH); Alexandru ZABARA, Kaiseraugst (CH)

(21) Appl. No.: 18/715,617

(22) PCT Filed: Nov. 29, 2022
(86) PCT No.: PCT/EP2022/083708

§ 371 Date: May 31, 2024

(30) Foreign Application Priority Data
Dec. 3, 2021 (EP) 21212297.2

(51) Int. Cl. A61K 31/375; A61K 9/50

(52) U.S. Cl. CPC A61K 31/375; A61K 9/5015; A61K 9/5036

(57) ABSTRACT

The present invention relates to a new delivery system for Vitamin C (also known as ascorbic acid and ascorbate) for the release large intestine. These nutritional ingredients are useful for gut and metabolic health in monogastric animals (such as swine and poultry as well as fish), especially in humans.

The present invention relates to a new delivery system for Vitamin C (also known as ascorbic acid and ascorbate) for release in the large intestine. These nutritional ingredients are useful for gut and metabolic health in monogastric animals (such as swine and poultry as well as fish), especially in humans.

In many cases, it is desirable for pharmaceutical and nutraceutical dosage units to pass through a stomach intact and release their contents upon reaching the intestines. This controlled release may be particularly desirable when an ingredient(s) of the dosage unit is unstable in the stomach's acidic environment and where the ingredient(s) is intended for release in the slightly alkaline conditions of the gastrointestinal tract beyond the stomach.

The new formulation according to the present invention allows releasing vitamin C in the large intestine.

Therefore, the present invention relates to a single coated delivery system (DS) consisting of
(a) a solid core, which comprises vitamin C, and
(b) a coating comprising
   shellac and/or shellac ammonium salt and
   at least one alginate having a viscosity of 10 to 800 mPas (10% aqueous solution at 20° C.),
   and at least one plasticiser.

The new delivery system is a single coated formulation. This means that the inner part of the formulation (the core) is covered by one single layer. The new delivery system does not comprise another additional layer.

Usually, the single coated delivery system according to the present invention comprises
60-95 weight-% (wt-%) (preferably 70-90 wt-%), based on the total weight of the single coated delivery system, of the core, and
5 to 40 wt-% (preferably 10-30 wt-%), based on the total weight of the single coated delivery system, of the coating layer.

Usually and preferably, the single coated delivery system consists of
60-95 wt-%, based on the total weight of the single coated delivery system, of the core, and
5 to 40 wt-%, based on the total weight of the single coated delivery system, of the coating layer.

More preferably, the single coated delivery system consists of
70-90 wt-%, based on the total weight of the single coated delivery system, of the core, and
10 to 30 wt-%, based on the total weight of the single coated delivery system, of the coating layer.

All percentages of the single coated delivery system always add up to 100.

Therefore, the present invention relates to a single coated delivery system (DS1), which is coated delivery system (DS), comprising 60-95 wt-%, based of the total weight of the single coated delivery system, of the core, and
5 to 40 wt-%, based on the total weight of the single coated delivery system, of the coating layer.

Therefore, the present invention relates to a single coated delivery system (DS1′), which is coated delivery system (DS), comprising 70-90 wt-%, based on the total weight of the single coated delivery system, of the core, and
10 to 30 wt-%, based on the total weight of the single coated delivery system, of the coating layer.

Therefore, the present invention relates to a single coated delivery system (DS1″), which is coated delivery system (DS), consisting of 60-95 wt-%, based of the total weight of the single coated delivery system, of the core, and
5 to 40 wt-%, based on the total weight of the single coated delivery system, of the coating layer.

Therefore, the present invention relates to a single coated delivery system (DS1″), which is coated delivery system (DS), consisting of 70-90 wt-%, based on the total weight of the single coated delivery system, of the core, and
10 to 30 wt-%, based on the total weight of the single coated delivery system, of the coating layer.

The coating of the single coated delivery system according to the present invention is covering the solid core completely. Ideally, the coating layer has a (more or less) homogenous thickness when applied on the solid core.

Usually, the thickness of the coating layer is at least 5 μm and not more than 50 μm. Preferably, the thickness of the coating layer is between 5 μm-50 μm.

Therefore, the present invention relates to a single coated delivery system (DS2), which is coated delivery system (DS), (DS1), (DS1′), (DS1″) or (DS1″), wherein the thickness of the coating layer (applied on the core) is at least 5 μm and not more than 50 μm.

Therefore, the present invention relates to a single coated delivery system (DS2′), which is coated delivery system (DS), (DS1), (DS1′), (DS1″) or (DS1′″), wherein the thickness of the coating layer is between 5 μm-50 μm.

The solid core of the single coated delivery system of the present invention comprises vitamin C.

The term “core” in the context of the present invention always refers to a particle, which is produced by any commonly known and used technology (such as spray-drying, spray-cooling, spray-granulation, spray-chilling, agglomeration, extrusion-spheronisation, pelletisation etc.) and which contains at least one active ingredient.

Vitamin C can be produced chemically or isolated from a natural source (primarily fruits and vegetables). For the formulation according to the present invention the source is not essential. Any vitamin C (also a mixture from various sources) can be used.

The core comprises up to 100 wt-% of vitamin C, based on the total weight of the core.

Therefore, the present invention relates to a single coated delivery system (DS3), which is coated delivery system (DS), (DS1), (DS1′), (DS1″), (DS1′″) or (DS2), wherein the core comprises up to 100 wt-% of vitamin C, based on the total weight of the core.

Furthermore, the core can comprise any commonly known auxiliary agents. These agents are usually used for the production of the core.

Such auxiliary agents are, for example, carriers (such as cellulose, maltodextrins, starch or lactose); binders (such as pectin, hydroxypropylmethylcellulose, gel-forming agents as xanthan gum, gellan gum); humectants (such as glycerine, sorbitol, polyethylene glycol, triethyl citrate); dyes; fragrances; fillers and buffers.

Such auxiliary agents are usually present in an amount up to 80 wt-%, based on the total weight of the core. (Usually 10 to 60 wt-%, preferably 10 to 40 wt-%, based on the total weight of the core).

Therefore, the present invention relates to a single coated delivery system (DS4), which is coated delivery system (DS), (DS1), (DS1′), (DS1″), (DS1′″), (DS2) or (DS3), wherein the core comprises at least one auxiliary agent.

Therefore, the present invention relates to a single coated delivery system (DS4′), which is coated delivery system (DS4), wherein the at least one auxiliary agent is chosen from the group consisting of carriers (such as cellulose, maltodextrins, starch or lactose); binders (such as pectin, hydroxypropylmethylcellulose, gel-forming agents as xanthan gum, gellan gum); humectants (such as glycerine, sorbitol, polyethylene glycol, triethyl citrate); dyes; fragrances; fillers and buffers.

Therefore, the present invention relates to a single coated delivery system (DS4″), which is coated delivery system (DS4) or (DS4′), wherein the core comprises up to 80 wt-% of at least one auxiliary agent, based on the total weight of the core.

Therefore, the present invention relates to a single coated delivery system (DS4′″), which is coated delivery system (DS4) or (DS4′), wherein the core comprises 10 to 60 wt-% of at least one auxiliary agent, based on the total weight of the core.

Therefore, the present invention relates to a single coated delivery system (DS4″″), which is coated delivery system (DS4) or (DS4′), wherein the core comprises 10 to 40 wt-% of at least one auxiliary agent, based on the total weight of the core.

The core of the single coated delivery system according to the present invention can be produced by any commonly known processes (such as spray-drying, spray-cooling, spray-granulation, spray-chilling, agglomeration, extrusion-spheronisation, pelletisation etc.).

The shape of the core is not an essential feature of the present invention. The shape can be sphere-like or any other form (also mixtures of shapes). Usually and preferably, the particles are sphere-like.

The size of the core as well as the size of the single coated delivery system according to the present invention, is not an essential feature of the present invention.

A suitable size of the single coated delivery system according to the present invention is between 100-2300 μm (preferably 300-2000 μm, more preferably 500-1400 μm); the size is defined by the diameter of the longest dimension of the particle and measured by a commonly known method (like laser diffraction or sieve analysis).

As stated above, the thickness of the coating layer is between 5 μm-50 μm.

Using a “Mastersizer 3000” of Malvern Instruments Ltd., UK, all particle sizes are determined by laser diffraction technique. Further information on this particle size characterisation method can e.g. be found in “Basic principles of particle size analytics”, Dr Alan Rawle, Malvern Instruments Limited, Enigma Business Part, Grovewood Road, Malvern, Worcestershire, WR14 1XZ, UK and the “Manual of Malvern particle size analyser”. Particular reference is made to the user manual number MAN 0096, Issue 1.0, November 1994. If nothing else is stated, all particle sizes referring are Dv90 values (volume diameter, 90% of the population resides below this point, and 10% resides above this point) determined by laser diffraction. The particle size can be determined in the dry form.

Therefore, the present invention relates to a single coated delivery system (DS5), which is a coated delivery system (DS), (DS1), (DS1′), (DS1′″), (DS1″″), (DS2), (DS3), (DS4), (DS4′), (DS4″), (DS4′″) or (DS4″″), wherein the size of the single coated delivery system is between 100-2300 μm.

Therefore, the present invention relates to a single coated delivery system (DS5′), which is a coated delivery system (DS), (DS1), (DS1′), (DS1″), (DS1′″), (DS2), (DS3), (DS4), (DS4′), (DS4″), (DS4″) or (DS4″), wherein the size of the single coated delivery system is between 300-2000 μm.

Therefore, the present invention relates to a single coated delivery system (DS5″), which is a coated delivery system (DS), (DS1), (DS1′), (DS1″), (DS1′″), (DS2), (DS3), (DS4), (DS4′), (DS4″), (DS4′″) or (DS4″″), wherein the size of the single coated delivery system is between 500-1400 μm.

The coating of the single coated delivery system according to the present invention comprises shellac and/or at least one shellac ammonium salt and at least one alginate having a viscosity of 10 to 800 mPas (10% aqueous solution at 20° C.) and at least one plasticiser.

This means that the coating of the single coated delivery system according to the present invention has at least three ingredients.

The first coating material is shellac and/or shellac ammonium salt.

Shellac is a natural product with interesting properties and exceptional versatility. Shellac is the purified product of the natural resin lac, which is the hardened secretion of the tiny, parasitic insect Kerria lacca, popularly known as the lac insect. It is the only known commercial resin of animal origin.

Shellac is a natural material with a complex mixture of esters and polyesters of polyhydroxy acids.

There are many commercial suppliers, which sell shellac.

Shellac ammonium salt is a modified shellac. This is done according to commonly known processes. Shellac ammonium salt is available commercially as well.

The shellac and/or the shellac ammonium salt is used in the single coating in an amount of 50 to 98 wt-%, based on the total weight of the single coating layer.

Preferably it is used in an amount of 60 to 85 wt-%, more preferably 70 to 85 wt-%, based on the total weight of the single coating layer.

Preferably shellac ammonium salt is used.

Therefore, the present invention relates to a single coated delivery system (DS6), which is coated delivery system (DS), (DS1), (DS1′), (DS1″), (DS1′″), (DS2), (DS3), (DS4), (DS4′), (DS4″), (DS4′″), (DS4″″), (DS5), (DS5′) or (DS5″), wherein shellac and/or the shellac ammonium salt is used in the single coating in an amount of 50 to 98 wt-%, based on the total weight of the single coating layer.

Therefore, the present invention relates to a single coated delivery system (DS6′), which is coated delivery system (DS), (DS1), (DS1′), (DS1″), (DS1′″), (DS2), (DS3), (DS4), (DS4′), (DS4″), (DS4′″), (DS4″″), (DS5), (DS5′) or (DS5″), wherein shellac and/or the shellac ammonium salt is used in the single coating in an amount of 60 to 85 wt-%, based on the total weight of the single coating layer.

Therefore, the present invention relates to a single coated delivery system (DS6″), which is coated delivery system (DS), (DS1), (DS1′), (DS1″), (DS1′″), (DS2), (DS3), (DS4), (DS4′), (DS4″), (DS4′″), (DS4″″), (DS5), (DS5′) or (DS5″), wherein shellac and/or the shellac ammonium salt is used in the single coating in an amount of 70 to 85 wt-%, based on the total weight of the single coating layer.

The single coating also comprises at least one alginate having a viscosity of 10 to 800 mPas (measured as 10% aqueous solution at 20° C.). The viscosity can be measured using all commonly known methods and apparatus.

Preferably the at least one alginate in the single coating has a viscosity of 10 to 500 mPas (measured as 10% solution at 20° C.).

More preferably the at least one alginate in the single coating has a viscosity of 10 to 200 mPas (measured as 10% solution at 20° C.).

Most preferably, the at least one alginate in the single coating has a viscosity of 10 to 100 mPas (measured as 10% solution at 20° C.).

Therefore, the present invention relates to a single coated delivery system (DS7), which is coated delivery system (DS), (DS1), (DS1′), (DS1″), (DS1′″), (DS2), (DS3), (DS4), (DS4′), (DS4″), (DS4′″), (DS4″″), (DS5), (DS5′), (DS5″), (DS6), (DS6′) or (DS6″), wherein the at least one alginate in the single coating has a viscosity of 10 to 800 mPas (measured as 10% aqueous solution at 20° C.).

Therefore, the present invention relates to a single coated delivery system (DS7′), which is coated delivery system (DS), (DS1), (DS1′), (DS1″), (DS1′″), (DS2), (DS3), (DS4), (DS4′), (DS4″), (DS4′″), (DS4″″), (DS5), (DS5′), (DS5″), (DS6), (DS6′) or (DS6″), wherein the at least one alginate in the single coating has a viscosity of 10 to 500 mPas (measured as 10% solution at 20° C.).

Therefore, the present invention relates to a single coated delivery system (DS7″), which is coated delivery system (DS), (DS1), (DS1′), (DS1″), (DS1′″), (DS2), (DS3), (DS4), (DS4′), (DS4″), (DS4′″), (DS4″″), (DS5), (DS5′), (DS5″), (DS6), (DS6′) or (DS6″), wherein the at least one alginate in the single coating has a viscosity of 10 to 200 mPas (measured as 10% solution at 20° C.).

Therefore, the present invention relates to a single coated delivery system (DS7″), which is coated delivery system (DS), (DS1), (DS1′), (DS1″), (DS1′″), (DS2), (DS3), (DS4), (DS4′), (DS4″), (DS4′″), (DS4″″), (DS5), (DS5′), (DS5″), (DS6), (DS6′) or (DS6″), wherein the at least one alginate in the single coating has a viscosity of 10 to 100 mPas (measured as 10% solution at 20° C.).

Such alginates are known as ultra-low-viscosity alginates.

Usually and preferably, it is a sodium alginate. Such ultra-low-viscosity alginates are available commercially from various suppliers (for example, KIMICA Corporation under the tradenames KIMICA ALGIN ULV-L3, KIMICA ALGIN ULV-L5, KIMICA ALGIN ULV-1, KIMICA ALGIN ULV-3 and KIMICA ALGIN ULV-5).

Therefore, the present invention relates to a single coated delivery system (DS8), which is coated delivery system (DS), (DS1), (DS1′), (DS1″), (DS1′″), (DS2), (DS3), (DS4), (DS4′), (DS4″), (DS4′″), (DS4″″), (DS5), (DS5′), (DS5″), (DS6), (DS6′), (DS6″), (DS7), (DS7′), (DS7″) or (DS7′″), wherein the at least one alginate in the single coating is a sodium alginate.

The at least one alginate is used in the single coating in an amount of 0.5 to 10 wt-%, based on the total weight of the single coating layer.

Preferably it is used in an amount of 1 to 6 wt-%, more preferably 2 to 5 wt-%, based on the total weight of the single coating layer.

Therefore, the present invention relates to a single coated delivery system (DS9), which is coated delivery system (DS), (DS1), (DS1′), (DS1″), (DS1′″), (DS2), (DS3), (DS4), (DS4′), (DS4″), (DS4′″), (DS4″″), (DS5), (DS5′), (DS5″), (DS6), (DS6′), (DS6″), (DS7), (DS7′), (DS7″), (DS7′″) or (DS8), wherein the at least one alginate in the single coating is used in an amount of 0.5 to 10 wt-%, based on the total weight of the single coating layer. Therefore, the present invention relates to a single coated delivery system (DS9′), which is coated delivery system (DS), (DS1), (DS1′), (DS1″), (DS1′″), (DS2), (DS3), (DS4), (DS4′), (DS4″), (DS4′″), (DS4″″), (DS5), (DS5′), (DS5″), (DS6), (DS6′), (DS6″), (DS7), (DS7′), (DS7″), (DS7′″) or (DS8), wherein the at least one alginate in the single coating is used in an amount of 1 to 6 wt-%, based on the total weight of the single coating layer.

Therefore, the present invention relates to a single coated delivery system (DS9″), which is coated delivery system (DS), (DS1), (DS1′), (DS1″), (DS1′″), (DS2), (DS3), (DS4), (DS4′), (DS4″), (DS4′″), (DS4″″), (DS5), (DS5′), (DS5″), (DS6), (DS6′), (DS6″), (DS7), (DS7′), (DS7″), (DS7′″) or (DS8), wherein the at least one alginate in the single coating is used in an amount of 2 to 5 wt-%, based on the total weight of the single coating layer.

The third component of the single coating layer is one or more plasticiser.

The plasticiser can be any commonly used ones. Suitable ones are e.g. glycols, glycerol, glycerol triacetate, polyethers, triethyl citrate.

Therefore, the present invention relates to a single coated delivery system (DS10), which is coated delivery system (DS), (DS1), (DS1′), (DS1″), (DS1′″), (DS2), (DS3), (DS4), (DS4′), (DS4″), (DS4′″), (DS4″″), (DS5), (DS5′), (DS5″), (DS6), (DS6′), (DS6″), (DS7), (DS7′), (DS7″), (DS7′″), (DS8), (DS9), (DS9′) or (DS9″), wherein the at least one plasticiser is chosen from the group consisting of glycols, glycerol, glycerol triacetate, polyethers, triethyl citrate.

The at least one plasticiser is used in the single coating in an amount of 1 to 30 wt-%, based on the total weight of the single coating layer.

Preferably it is used in an amount of 5 to 30 wt-%, more preferably 10 to 25 wt-%, based on the total weight of the single coating layer.

Therefore, the present invention relates to a single coated delivery system (DS11), which is coated delivery system (DS), (DS1), (DS1′), (DS1″), (DS1′″), (DS2), (DS3), (DS4), (DS4′), (DS4″), (DS4′″), (DS4″″), (DS5), (DS5′), (DS5″), (DS6), (DS6′), (DS6″), (DS7), (DS7′), (DS7″), (DS7′″), (DS8), (DS9), (DS9′), (DS9″) or (DS10), wherein the at least one plasticiser in the single coating is used in an amount of 1 to 30 wt-%, based on the total weight of the single coating layer.

Therefore, the present invention relates to a single coated delivery system (DS11′), which is coated delivery system (DS), (DS1), (DS1′), (DS1″), (DS1′″), (DS2), (DS3), (DS4), (DS4′), (DS4″), (DS4′″), (DS4″″), (DS5), (DS5′), (DS5″), (DS6), (DS6′), (DS6″), (DS7), (DS7′), (DS7″), (DS7′″), (DS8), (DS9), (DS9′), (DS9″) or (DS10), wherein the at least one plasticiser in the single coating is used in an amount of 5 to 30 wt-%, based on the total weight of the single coating layer.

Therefore, the present invention relates to a single coated delivery system (DS11″), which is coated delivery system (DS), (DS1), (DS1′), (DS1″), (DS1′″), (DS2), (DS3), (DS4), (DS4′), (DS4″), (DS4′″), (DS4″″), (DS5), (DS5′), (DS5″), (DS6), (DS6′), (DS6″), (DS7), (DS7′), (DS7″), (DS7′″), (DS8), (DS9), (DS9′), (DS9″) or (DS10), wherein the at least one plasticiser in the single coating is used in an amount of 10 to 25 wt-%, based on the total weight of the single coating layer.

It is clear that all percentages of the ingredients of the single coating layer are always added up to 100.

The single coated delivery system according to the present invention are produced according to commonly known and used processes, such as fluid-bed coating, Wurster coating, bottom-spray coating, tangential spray coating.

The new delivery systems (DS), (DS1), (DS1′), (DS1″), (DS1′″), (DS2), (DS3), (DS4), (DS4′), (DS4″), (DS4′″), (DS4″″), (DS5), (DS5′), (DS5″), (DS6), (DS6′), (DS6″), (DS7), (DS7′), (DS7″), (DS7′″), (DS8), (DS9), (DS9′), (DS9″), (DS10), (DS11), (DS11′) and/or (DS11″) according to the present invention can be used as such or incorporated into any application forms.

The new delivery systems (DS), (DS1), (DS1′), (DS1″), (DS1′″), (DS2), (DS3), (DS4), (DS4′), (DS4″), (DS4′″), (DS4″″), (DS5), (DS5′), (DS5″), (DS6), (DS6′), (DS6″), (DS7), (DS7′), (DS7″), (DS7′″), (DS8), (DS9), (DS9′), (DS9″), (DS10), (DS11), (DS11′) and/or (DS11″) can be used as such or in any dietary supplement, food product, feed product, personal care product or pharmaceutical product.

The new delivery systems (DS), (DS1), (DS1′), (DS1″), (DS1′″), (DS2), (DS3), (DS4), (DS4′), (DS4″), (DS4′″), (DS4″″), (DS5), (DS5′), (DS5″), (DS6), (DS6′), (DS6″), (DS7), (DS7′), (DS7″), (DS7′″), (DS8), (DS9), (DS9′), (DS9″), (DS10), (DS11), (DS11′) and/or (DS11″) can also be part of a premix formulation, which can then be used to formulate any dietary supplement, food product, feed product, personal care product or pharmaceutical product.

The invention also relates to a process for the production of a premix, dietary supplement, food product, feed product, personal care product or pharmaceutical product using at least one delivery system (DS), (DS1), (DS1′), (DS1″), (DS1′″), (DS2), (DS3), (DS4), (DS4′), (DS4″), (DS4′″), (DS4″″), (DS5), (DS5′), (DS5″), (DS6), (DS6′), (DS6″), (DS7), (DS7′), (DS7″), (DS7′″), (DS8), (DS9), (DS9′), (DS9″), (DS10), (DS11), (DS11′) and/or (DS11″).

The invention also relates to a premix, dietary supplement, food product, feed product, personal care product or pharmaceutical product comprising at least one delivery systems (DS), (DS1), (DS1′), (DS1″), (DS1′″), (DS2), (DS3), (DS4), (DS4′), (DS4″), (DS4′″), (DS4″″), (DS5), (DS5′), (DS5″), (DS6), (DS6′), (DS6″), (DS7), (DS7′), (DS7″), (DS7′″), (DS8), (DS9), (DS9′), (DS9″), (DS10), (DS11), (DS11′) and/or (DS11″).

The following examples serve to illustrate specific embodiments of the invention claimed herein. All percentages are given in relation to the weight, and all the temperatures are given in degrees Celsius.

### EXAMPLES

### Example 1

For the spray solution, 0.13 g Na-alginate (Kimica Algin ULV-L3, Kimica, Japan) and 0.9 g glycerol (Sigma Aldrich, Switzerland) was dissolved in 66.4 g shellac ammonium salt solution (25% solids, Aquagold SSB, Stroever, Germany) with gentle stirring.

100 g Core granulate containing 68% ascorbic acid was placed in a lab-scale fluid-bed processor (WFP mini, DMR, Switzerland) with bottom-spray configuration and fluidised. Inlet air temperature was set to 80° C. The shellac-alginate-glycerol solution was sprayed on the core pellets. The spray rate was set to keep a product temperature of 49-59° C. After coating, the product was dried in the fluid bed at 45-52° C. 114 g coated product was obtained.

The coating layer contains 94.2% shellac ammonium salt, 0.7% Na-alginate and 5.1% glycerol as the plasticiser.

| | single coating with | Comparative example |
|---|---|---|
| | 0.75% alginate in | 5% alginate, |
| % cumulative | shellac | 1% CaCl₂ 15% shellac |
| release | (Example 1) | (Comp. Exp) |
| stomach | 2 | 1 |
| proximal small | 3 | 2 |
| intestine | | |
| ileum | 7 | 3 |
| Colon 1 h | 79 | 72 |
| Colon 8 h | 96 | 78 |
| Colon 18 h | 97 | 79 |

1. A single coated delivery system consisting of
(a) a solid core, which comprises vitamin C, and
(b) a coating comprising
shellac and/or shellac ammonium salt and
at least one alginate having a viscosity of 10 to 800 mPas (10% aqeuous solution at 20° C.) and
at least one plasticiser.

2. Single coated delivery system according to claim 1 consisting of 60-95 wt-%, based on the total weight of the single coated delivery system, of the core, and 5 to 40 wt-%, based on the total weight of the single coated delivery system, of the coating layer.

3. Single coated delivery system according to claim 1 consisting of 70-90 wt-%, based on the total weight of the single coated delivery system, of the core, and 10 to 30 wt-%, based on the total weight of the single coated delivery system, of the coating layer.

4. Single coated delivery system according to claim 1, wherein the thickness of the coating layer is at least 5 μm.

5. Single coated delivery system according to claim 1, wherein the thickness of the coating layer is between 5 μm-50 μm.

6. Single coated delivery system according to claim 1, wherein the core comprises up to 100 wt-% of vitamin C, based on the total weight of the core.

7. Single coated delivery system according to claim 1, wherein the size of the single coated delivery system is between 100-2300 μm.

8. Single coated delivery system according to claim 1, wherein shellac and/or the shellac ammonium salt is used in the single coating in an amount of 50 to 98 wt-%, based on the total weight of the single coating layer.

9. Single coated delivery system according to claim 1, wherein the at least one alginate in the single coating has a viscosity of 10 to 800 mPas (measured as 10% solution at 20° C.).

10. Single coated delivery system according to claim 1, wherein the at least one alginate is a sodium alginate.

11. Single coated delivery system according to claim 1, wherein the at least one alginate in the single is used in the single coating in an amount of 0.5 to 10 wt-%, based on the total weight of the single coating layer.

12. Single coated delivery system according to claim 1, wherein the at least one plasticiser is chosen from the group consisting of glycols, glycerol triacetate, polyethers, triethyl citrate.

13. Single coated delivery system according to claim 1, wherein the at least one plasticiser in the single coating is used in an amount of 1 to 30 wt-%, based on the total weight of the single coating layer.
